(19) United States
(12) Patent Application Publication
(10) Pub. No.: US 2023/0094761 A1
(43) Pub. Date: Mar. 30, 2023

(54) SYSTEM AND METHOD FOR CULINARY GAMIFICATION

(71) Applicant: Grace Choi, Reston, VA (US)

(72) Inventor: Grace Choi, Reston, VA (US)

(21) Appl. No.: 18/075,452

(22) Filed: Dec. 6, 2022

Related U.S. Application Data
(63) Continuation of application No. 16/598,923, filed on Oct. 10, 2019.
(60) Provisional application No. 62/743,586, filed on Oct. 10, 2018.

(51) Int. Cl. G09B 19/00; G11B 27/22; G06F 3/0488; A47J 36/32; G16H 20/60; A47J 27/00

(52) U.S. Cl. CPC G09B 19/0092; G11B 27/22; G06F 3/0488; A47J 36/321; G16H 20/60; A47J 27/004

(57) ABSTRACT

Systems and methods are provided for facilitating food preparation. The method includes opening, using an electronic device, a digital application, creating a user profile with the digital application, selecting, using a graphical user interface, one or more grocery stores to be designated grocery stores, selecting, using the graphical user interface, a recipe for preparation by a user, displaying, using the graphical user interface, a list of one or more ingredients required for preparation of the recipe, selecting, using the graphical user interface in conjunction with the digital application, one or more items in the list of the one or more ingredients required for preparation of the recipe, ordering, using the digital application, any items selected from the one or more items in the list of the one or more ingredients required for preparation of the recipe, and displaying, to the user, using the graphical user interface, the recipe.

### CLAIM OF PRIORITY

This application is a continuation of U.S. Non-Provisional application Ser. No. 16/598,923, filed on Oct. 10, 2019 entitle SYSTEM AND METHOD FOR CULINARY GAMIFICATION which claims the priority from U.S. provisional application Ser. No. 62/743,586, filed Oct. 10, 2019 entitled SYSTEM AND METHOD FOR CULINARY GAMIFICATION, the entirety of which is hereby incorporated by reference as if fully set forth herein.

### FIELD OF THE EMBODIMENTS

This invention relates to food preparation and, particularly, to preparing one or more dishes using a digital application.

### Background of the Embodiments

Although there are many options for purchasing pre-prepared food, there remains a need and desire among many people to prepare their own dishes. This preparation, however, is not always an easy task. Preparing food dishes can be an arduous, involving many steps from determining ingredients, to determining materials, to purchasing any needed ingredients or materials, to determining the different steps to prepare the dish.

Oftentimes, these various tasks can be very time consuming and costly, especially for people who are uncertain where to find certain ingredients or are unclear about which brand or type of ingredients to buy.

For at least these reasons, a system and method in which users can be aided in the preparation of food dishes and in the procurement of any necessary ingredients and materials is needed.

Examples of related art are described below:

U.S. Pat. No. 8,655,674 generally describes a method of providing personal service assistance in a virtual universe. The method provides a customer avatar (CA) access to a personal assistance avatar or personal customer service representative (PCSR) avatar, hereinafter PCSR avatar. Through the PCSR avatar, the CA communicates a need or requests for personal service assistance from a business or organization. The communication of the received request is processed to identify an appropriate business, organization or vendor that meets the need or request. The identified business, organization or vendor is contacted for a representative from the business to accede to the request from the CA. The representative from the business is allowed to assume control of the PCSR avatar to conduct the session necessary for rendering the assistance or transaction. Upon completion of the session, control of the PCSR avatar is reverted to that prior to the session.

U.S. Pat. No. 9,576,334 generally describes systems for, and methods of, displaying video information comprising: a second screen device obtaining current play position data of a video being played on a primary screen device (e.g., obtaining from the primary screen device an identification of a current play position of the video, or obtaining information to generate an acoustic fingerprint of the video); determining a current play position of the video playing on the primary screen device based upon the current play position data (e.g., identification of the current play position or the acoustic fingerprint); downloading information (e.g., video map, subtitles, moral principles, objectionable content, memorable content, performers, geographical maps, shopping, plot point, item, ratings, trivia information, and recipe information) over a computer communications network into the memory of the second screen device; and displaying information on the second screen device synchronized with the contemporaneously played video on the primary screen device.

U.S. Pat. No. 9,805,381 generally describes computation of a preference score for a certain type of food. In some embodiments described herein, measurements of affective response of at least ten users are collected. The measurements may include various values indicative of physiological signals and/or behavioral cues of the at least ten users. Each measurement of a user is taken with a sensor coupled to the user up to four hours after the user consumed the certain type of food. A preference score is computed based on the measurements. The preference score is indicative of how much the at least ten users enjoyed consuming the certain type of food and/or how well they felt after consuming the certain type of food.

U.S. Patent Publication No. 2012/0231437 generally describes an online course and learning material management system and methods where students, instructors, publishers, and industry partners utilize systems' databases and networking features to interact and improve learning outcome. The system furthermore allows for networking among students, instructors, publishers, software vendors, and prospective employers.

U.S. Patent Publication No. 2013/0130217 generally describes e-learning lesson delivery platforms, products, programs, and methods comprising a digital processing device and a program that creates a lesson delivery server, wherein said server comprises: a plurality of learning activities, wherein said activities are organized according to an instructional plan designed to accomplish one or more educational objectives in at least one Subject, wherein said plan identifies one or more activities for use in a guided environment and one or more activities for assignment as independent work; a module for displaying and providing access to said one or more activities in a guided environment; a module for assigning said one or more activities as independent work to one or more learners, wherein said module is only accessible by a mentor, and a module for displaying and providing access to activities assigned as independent work, wherein said module is accessible by a mentor or a learner.

U.S. Patent Publication No. 2016/0140244 generally describes a method including a plurality of client applications providing an electronic interface for receiving personality information and conversation preference information of corresponding users, wherein each client application of the plurality of client applications runs on a corresponding computing platform. The method includes one or more applications running on a processor of a remote server receiving the personality information and conversation preference information from the plurality of client applications, the one or more applications using the personality information and conversation preference information to match a first user of a first client application with a second user of a second client application. The method includes the one or more applications establishing a communications coupling between the first client application and the second client application, wherein the communications coupling comprises a communication session.

U.S. Patent Publication No. 2016/0188738 generally describes a system/method in which a user request is received from a mobile client device, where the user request includes at least a speech input and seeks an informational answer or performance of a task. A failure to provide a satisfactory response to the user request is detected. In response to detection of the failure, information relevant to the user request is crowd-sourced by querying one or more crowd sourcing information sources. One or more answers are received from the crowd sourcing information sources, and the response to the user request is generated based on at least one of the one or more answers received from the one or more crowd sourcing information sources.

U.S. Patent Publication No. 2017/0290454 generally describes a method of preparing meals in a computerized kitchen workspace is set forth. The computerized kitchen workspace includes one or more robots for preparing and cooking food in the kitchen workspace. Kitchen appliances may be automatically controlled according to a recipe. The one or more robots may access kitchen items necessary for meal preparation by rotating automated shelves. The meal may then be automatically prepared by the one or more robots. Automated storage shelves may be provided with electrical power for powering kitchen appliances found on the shelves.

U.S. Patent Publication No. 2017/0323640 generally describes systems and methods for obtaining content over the Internet, identifying text within the content (e.g., such as closed captioning or recipe text) or creating text from the content using such technologies as speech recognition, analyzing the text for actionable directions, and translating those actionable directions into instructions suitable for network-connected cooking appliances. Certain embodiments provide additional guidance to avoid or correct mistakes in the cooking process, and allow for the customization of recipes to address, e.g., dietary restrictions, culinary preferences, translation into a foreign language, etc.

None of the art described above addresses all of the issues that the present invention does.

### Summary of the Embodiments

According to an aspect of the present invention, a method for facilitating food preparation is provided. The method includes opening, using an electronic device having a graphical user interface, a digital application, creating, using the graphical user interface, a user profile with the digital application, selecting, using the graphical user interface, one or more grocery stores to be designated grocery stores, selecting, using the graphical user interface, a recipe for preparation by a user, displaying, using the graphical user interface, a list of one or more ingredients required for preparation of the recipe, selecting, using the graphical user interface in conjunction with the digital application, one or more items in the list of the one or more ingredients required for preparation of the recipe, ordering, using the digital application, any items selected from the one or more items in the list of the one or more ingredients required for preparation of the recipe, and displaying, to the user, using the graphical user interface, the recipe.

It is an object of the present invention to provide the method for facilitating food preparation, wherein the ordering further includes ordering from one or more of the designated grocery stores.

It is an object of the present invention to provide the method for facilitating food preparation, wherein the selecting the recipe further includes selecting the recipe from a list of available recipes.

It is an object of the present invention to provide the method for facilitating food preparation, wherein the method further includes searching, using the graphical user interface, through the list of available recipes.

It is an object of the present invention to provide the method for facilitating food preparation, wherein the electronic device is selected from the group consisting of: a smart phone; a desktop computer; a laptop computer; and a tablet computer.

It is an object of the present invention to provide the method for facilitating food preparation, wherein the displaying the recipe further includes reading, using a speaker, each step in preparing the recipe.

It is an object of the present invention to provide the method for facilitating food preparation, wherein the ordering further includes selecting which store to purchase each item in the items selected from the one or more items in the list of the one or more ingredients required for preparation of the recipe.

It is an object of the present invention to provide the method for facilitating food preparation, wherein the method further includes delivering, to the user, the items selected from the one or more items in the list of the one or more ingredients required for preparation of the recipe.

It is an object of the present invention to provide the method for facilitating food preparation, wherein the ordering further includes sending, to one or more grocery stores, a list of the items selected from the one or more items in the list of the one or more ingredients required for preparation of the recipe.

It is an object of the present invention to provide the system for facilitating food preparation, wherein the electronic device is further configured to enable the user to search, using the graphical user interface, voice user interface, and/or haptic interface, through the list of available recipes, technique videos, glossary terminologies, and narrative media.

It is an object of the present invention to provide the system for facilitating food preparation, wherein the electronic device is selected from the group consisting of a smart phone; a desktop computer; a laptop computer; a tablet computer; a smart watch, and a smart speaker.

According to another aspect of the present invention, a system for facilitating food preparation is provided. The system includes an electronic device, wherein the electronic device includes a memory, a processor, and a graphical user interface having a display. The electronic device is configured to open, using an electronic device having a graphical user interface, a digital application, create, using the processor, a user profile with the digital application using information input using the graphical user interface, receive a selection of one or more grocery stores to be designated grocery stores, receive a selection of a recipe for preparation by a user using the graphical user interface, display, using the graphical user interface, a list of one or more ingredients required for preparation of the recipe, receive a selection of one or more items in the list of the one or more ingredients required for preparation of the recipe, order any items selected from the one or more items in the list of the one or more ingredients required for preparation of the recipe, and display, to the user, using the graphical user interface, the recipe.

It is an object of the present invention to provide the system for facilitating food preparation, wherein the ordering further includes ordering from one or more of the designated grocery stores.

It is an object of the present invention to provide the system for facilitating food preparation, wherein the receiving the selection of the recipe further includes receiving the selection of the recipe from a list of available recipes.

It is an object of the present invention to provide the system for facilitating food preparation, wherein the electronic device is further configured to enable the user to search, using the graphical user interface, through the list of available recipes.

It is an object of the present invention to provide the system for facilitating food preparation, wherein the electronic device is selected from the group consisting of: a smart phone; a desktop computer; a laptop computer; and a tablet computer.

It is an object of the present invention to provide the system for facilitating food preparation, wherein the electronic device further includes a speaker configured to read each step in preparing the recipe.

It is an object of the present invention to provide the system for facilitating food preparation, wherein the electronic device is further configured to receive a selection of which store to purchase each item in the items selected from the one or more items in the list of the one or more ingredients required for preparation of the recipe.

It is an object of the present invention to provide the system for facilitating food preparation, wherein the system further includes one or more remote servers.

It is an object of the present invention to provide the system for facilitating food preparation, wherein the electronic device is coupled to the one or more remote servers.

It is an object of the present invention to provide the system for facilitating food preparation, wherein one or more second electronic devices are coupled to the one or more remote servers.

It is an object of the present invention to provide the system for facilitating food preparation, wherein the one or more servers are configured to send, to one or more grocery stores, a list of the items selected from the one or more items in the list of the one or more ingredients required for preparation of the recipe.

It is an object of the present invention to provide the system for enhancing food preparation skills, wherein the user is able to track activity, progress, and user-reported assessments of recipes and content, as well as life-log activities on social media.

It is an object of the present invention to provide the system for facilitating food preparation, wherein the method further includes socialization within and around the recipe delivery experience with other users, including commentary on recipe modifications, reviews, techniques, and tips.

It is an object of the present invention to provide the system for facilitating food preparation, wherein the method further includes offering recipe and media recommendations to the user based on user-generated feedback.

It is an object of the present invention to provide the system for facilitating food preparation, wherein the method further includes user-driven modifications and alternative pathways around the recipes based on dietary, ingredient, or material needs.

It is an object of the present invention to provide the system for facilitating food preparation, wherein the method further includes the execution of multiple recipes simultaneously.

### BRIEF DESCRIPTION OF THE DRAWINGS

FIGS. 1-2 show a flowchart of a method for preparing one or more dishes using a digital application, according to an embodiment of the present invention.

FIG. 3 shows a system for preparing one or more dishes using a digital application, according to an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

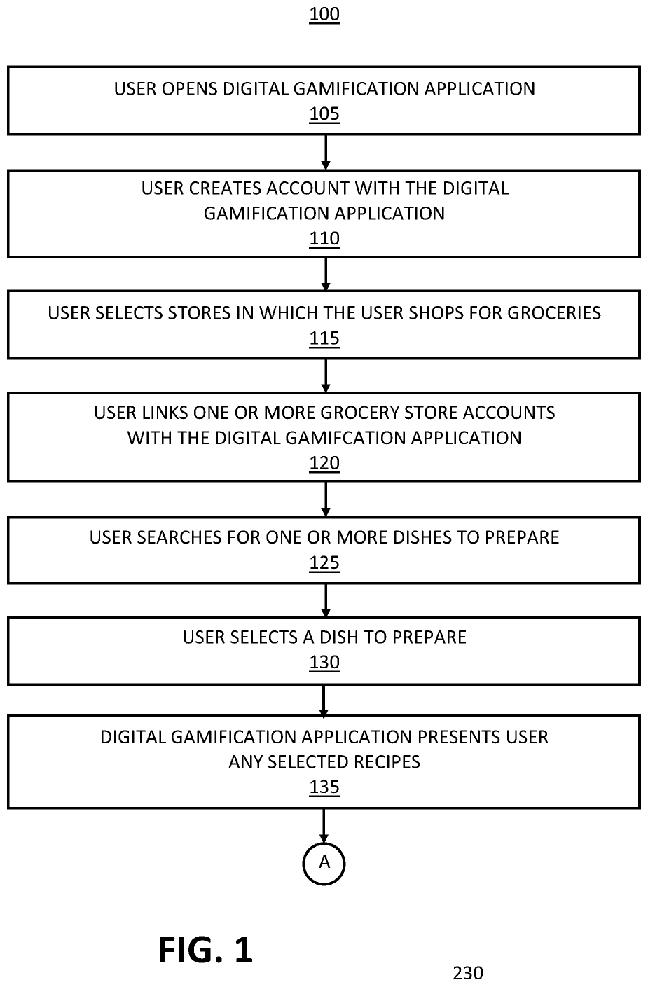

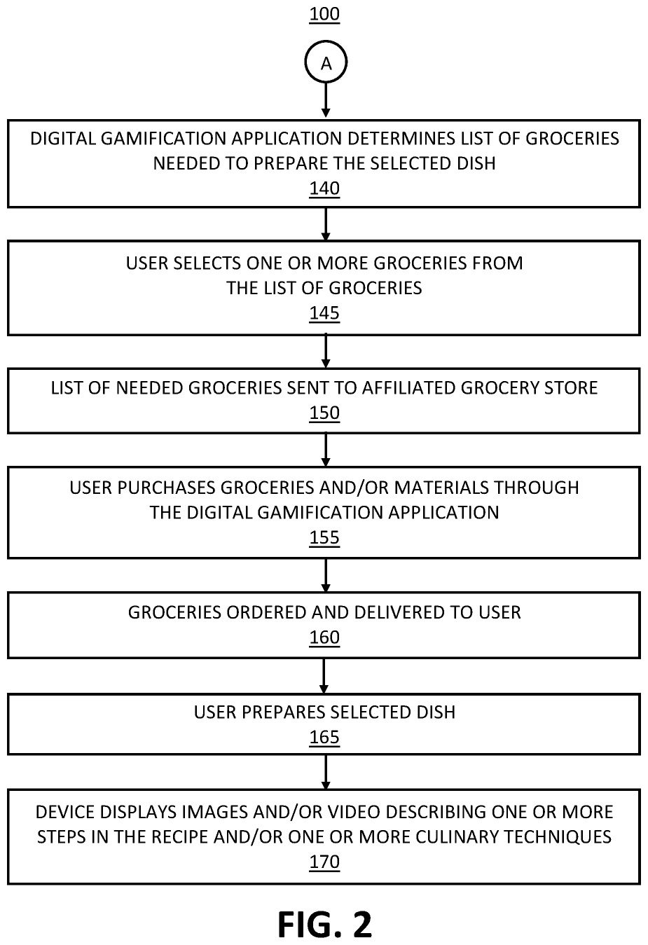

Referring now to FIGS. 1-2, a method 100 for preparing one or more dishes using a digital application is illustratively depicted, in accordance with an embodiment of the present invention.

Food preparation can range from very simple recipes calling for few and/or easy-to-understand ingredients and/or materials, to complicated ingredient lists requiring extensive preparation and precise measurements. Many people wish to be able to prepare food dishes along this spectrum. The present invention aids individuals in procuring the ingredients and materials needed and in preparing the food dishes, step by step.

At step 105, a user opens a digital gamification application on an electronic device. According to an embodiment, the electronic device may be, e.g., a desktop computer, a laptop computer, a tablet computer, a smart phone, and/or any other suitable electronic device.mn

At step 110, the user creates an account with the digital application. According to an embodiment, the account creation includes inputting identifiable information about the user. The identifiable information may include, e.g., a name/username, an e-mail address, payment information, a language for communication, age, gender, geographic location, race/ethnicity, skill level with cooking, cooking interests, and/or any other suitable information. According to an embodiment, the cooking interests may include the reason for cooking and/or using the digital application, who the user's favorite chefs are, the user's favorite cuisines to cook and/or eat, the user's favorite dining establishments, the user's favorite cooking channels/publishers/writers/etc., and/or any other suitable information regarding the user's cooking interests.

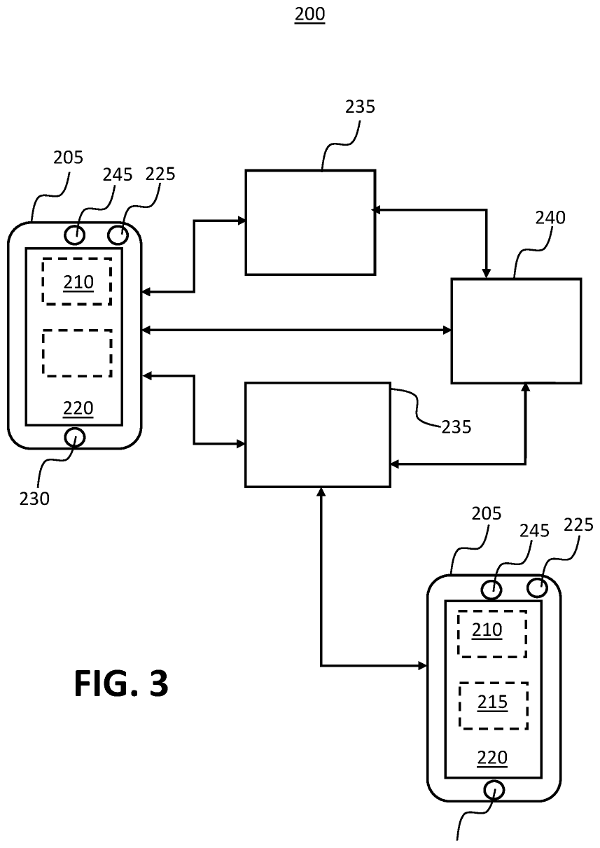

According to an embodiment, the user opens the application using a digital electronic device 205 (as shown in system 200 in FIG. 3). According to an embodiment, the electronic device 205 may be a smart phone. However, it is noted that any suitable electronic device may be used such as, but not limited to, a desktop computer, a laptop computer, and a tablet computer. According to an embodiment, the electronic device 205 includes a memory 210, a processor 215, and a graphical user interface 220, which may include an integral or secondary display. According to an embodiment, the electronic device further includes a camera 225, an audio player 245 (e.g., a speaker), and/or an audio recording device 230 (e.g., a microphone).

At step 115, the user, using the graphical user interface 220, selects one or more stores in which the user shops for groceries. According to an embodiment, if the user has a delivery account with the one or more grocery stores, the user, at step 120, links the grocery delivery account with the digital application.

According to an embodiment, the user, at step 125, searches for a dish to prepare. According to an embodiment, the user is able to search for dishes by name, source, ingredient(s), theme, diet, chef, degree of difficulty, occasion, and/or any other suitable criteria. According to an embodiment, the digital application suggests one or more dishes for the user to select. At step 130, the user selects a dish that he/she would like to prepare.

At step 135, the digital application presents the user with any selected recipes. According to an embodiment. According to an embodiment, the user is presented with the ingredients for the selected recipe on a display. The display may list the names, sources, and/or images of the ingredients. According to an embodiment, the display further show the procedures and steps for preparing the recipe, any comments/reviews for the recipe, a shopping list for the recipe, a material list for the recipe, and ancillary content (videos, advertisements, etc.) for the recipe, and/or any other suitable information pertaining to the recipe. According to an embodiment, the ingredients and/or materials list and/or instructions are read to the user over a speaker using a digital and/or prerecorded voice.

At step 140, the digital application determines a list of groceries that the user needs to prepare the selected dish. Once the user views the list of groceries needed for the selected recipe, the user, at step 145, may select one or more of the ingredients needed. According to an embodiment, the user may, at step 150, send the list of needed groceries to an affiliated grocery store. According to an embodiment, the user may also print, send, and/or share the list of needed groceries.

According to an embodiment, the user, at step 155, may purchase the groceries and/or materials through the digital application. According to an embodiment, the user may be provided with one or more brands to choose from for each ingredient and/or material. According to an embodiment, the quantities are preselected by the digital application so that the user has enough ingredients to prepare the selected dish. According to an embodiment, the user is able to select the grocery store(s) from which the groceries and/or materials will be purchased.

According to an embodiment, the digital application, at step 160, orders the ingredients for the food from the grocery stores using the linked user grocery delivery accounts and the groceries are delivered to the user. According to an embodiment, the user purchases his/her own groceries in order to prepare the selected dish. According to an embodiment, the order is delivered to an address supplied by the user. By facilitating the purchase of the proper materials and ingredients while instructing users on how to prepare food dishes, the present invention is an improvement upon the existing technologies.

According to an embodiment, the electronic device 205 is connected to one or more remote servers 235. According to an embodiment, the server 205 is coupled to the electronic device 205 via a wired and/or wireless connection. According to an embodiment, the server 235 stores some or all of the information input by the user using the graphical user interface 220. According to an embodiment, the server 235 is interactively coupled to the electronic device 205 and the one or more grocery or other stores 240. According to an embodiment, two or more electronic devices 205 are coupled to the one or more remote servers, enabling the two or more electronic devices 205 to communicate with each other.

At step 165, once all ingredients and/or materials are procured, the user prepares the selected dish. According to an embodiment, the electronic device, at step 170, shows the user the steps and/or reads off (using a smart speaker 245) the steps to prepare the selected dish. For example, the smart speaker may read off the ingredients needed, the materials needed, how to perform each step in the recipe, the length of time each step will approximately take for each step, how much of each ingredient is needed for each step, and/or any other relevant information pertaining preparing the recipe for the selected dish. According to an embodiment, step 170 comprises a device that displays images, video, and/or speech describing one or more steps in the recipe and/or one or more culinary techniques and/or one or more forms of audio or visual entertainment content.

According to an embodiment, a user, via the system, opens a digital gamification application; the user creates an account, the user specifies preferences (cuisines, personalities, skill-level, diets, usage level); the user selects stores; the user links their grocery accouns; the user searches for one or more dishes to prepare; the user selects recipe pathways should alternate pathways exist; and the system configures user with a digital recipe gamification. According to an embodiment the method and processes of the system are nonlinear and various options for different steps may be configured.

According to an embodiment, the electronic device displays images and/or a video describing one or more steps in the recipe and/or one or more culinary techniques used in preparing the selected dish. According to an embodiment, using the display, a microphone, and/or one or more speakers, the user may interact with other users preparing the recipe using the digital application.

According to an embodiment, the user may save recipes for others to use. According to an embodiment, the user may record (video and/or audio) himself/herself preparing a recipe and incorporate these recordings with the recipe.

According to an embodiment, the application is configured to enable users to socialize with other users, enabling the formation of communities and sub-communities within the application to engage in discourse around recipes and their execution. According to an embodiment, this includes uploading imagery of various steps, marking images and video as “favorites”, and being given tips and suggestions that cohere with their preferences, among other features. According to an embodiment, the application is configured to enable the users to upload images of their own cooking steps. According to an embodiment, other users are able to access the user-uploaded images. According to an embodiment, user-uploaded images are shown with the user who uploaded the images identified.

According to an embodiment, the acquisition of grocery and materials using the system is achievable. This is an ancillary feature that is optional. The preferred embodiment is a system configured to deliver a multi-modal guided recipe experience that facilitates food preparation while also providing a form of culinary entertainment.

In an embodiment, the graphical user interface shall also comprise a voice user interface and a haptic interface. In an embodiment, electronic devices shall also comprise standalone virtual assistants such as Amazon Echo or Echo Show, smart watches, and other smart devices.

According to an embodiment, the system may be gamified and inclusive of tracking and life-logging progress. For example, socializing around the recipe (this is if home cooks want to be able to cook dishes at the same time as others—friends or otherwise—remotely, or offer their own recommendations or modifications) can be achieved. According to an embodiment, recipe recommendations can be configured. Such recommendation can be self-driven modifications wherein one recipe may have several different pathways depending on factors determined by the user (access to materials and ingredients, dietary preferences). The system may be configured to execute layered recipe, wherein in multiple recipes can be prepared simultaneously.

Systems, Devices and Operating Systems

Typically, a user or users, which may be people or groups of users and/or other systems, may engage information technology systems (e.g., computers) to facilitate operation of the system and information processing. In turn, computers employ processors to process information and such processors may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the present invention may be connected to and/or communicate with entities such as, but not limited to: one or more users from user input devices; peripheral devices; an optional cryptographic processor device; and/or a communications network. For example, the present invention may be connected to and/or communicate with users, operating client device(s), including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™ HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The present invention may be based on computer systems that may comprise, but are not limited to, components such as: a computer systemization connected to memory.

Computer Systemization

A computer systemization may comprise a clock, central processing unit (“CPU(s)” and/or “processor(s)” (these terms are used interchangeable throughout the disclosure unless noted to the contrary)), a memory (e.g., a read only memory (ROM), a random access memory (RAM), etc.), and/or an interface bus, and most frequently, although not necessarily, are all interconnected and/or communicating through a system bus on one or more (mother)board(s) having conductive and/or otherwise transportive circuit pathways through which instructions (e.g., binary encoded signals) may travel to effect communications, operations, storage, etc. Optionally, the computer systemization may be connected to an internal power source; e.g., optionally the power source may be internal. Optionally, a cryptographic processor and/or transceivers (e.g., ICs) may be connected to the system bus. In another embodiment, the cryptographic processor and/or transceivers may be connected as either internal and/or external peripheral devices via the interface bus I/O. In turn, the transceivers may be connected to antenna(s), thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing the controller of the present invention to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the present invention and beyond through various interfaces. Should processing requirements dictate a greater amount speed and/or capacity, distributed processors (e.g., Distributed embodiments of the present invention), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the present invention may be achieved by implementing a microcontroller such as CAST's R8051XC2 microcontroller; Intel's MCS 51 (i.e., 8051 microcontroller); and/or the like. Also, to implement certain features of the various embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the component collection (distributed or otherwise) and/or features of the present invention may be implemented via the microprocessor and/or via embedded components; e.g., via ASIC, coprocessor, DSP, FPGA, and/or the like. Alternately, some implementations of the present invention may be implemented with embedded components that are configured and used to achieve a variety of features or signal processing.

Depending on the particular implementation, the embedded components may include software solutions, hardware solutions, and/or some combination of both hardware/software solutions. For example, features of the present invention discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the features of the present invention. A hierarchy of programmable interconnects allow logic blocks to be interconnected as needed by the system designer/administrator of the present invention, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the present invention may be developed on regular FPGAs and then migrated into a fixed version that more resembles ASIC implementations. Alternate or coordinating implementations may migrate features of the controller of the present invention to a final ASIC instead of or in addition to FPGAs. Depending on the implementation all of the aforementioned embedded components and microprocessors may be considered the “CPU” and/or “processor” for the present invention.

Power Source

The power source may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell is connected to at least one of the interconnected subsequent components of the present invention thereby providing an electric current to all subsequent components. In one example, the power source is connected to the system bus component. In an alternative embodiment, an outside power source is provided through a connection across the I/O interface. For example, a USB and/or IEEE 1394 connection carries both data and power across the connection and is therefore a suitable source of power.

Interface Adapters

Interface bus(ses) may accept, connect, and/or communicate to a number of interface adapters, conventionally although not necessarily in the form of adapter cards, such as but not limited to: input output interfaces (I/O), storage interfaces, network interfaces, and/or the like. Optionally, cryptographic processor interfaces similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces may accept, communicate, and/or connect to a number of storage devices such as, but not limited to: storage devices, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces may accept, communicate, and/or connect to a communications network. Through a communications network, the controller of the present invention is accessible through remote clients (e.g., computers with web browsers) by users. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed embodiments of the present invention), architectures may similarly be employed to pool, load balance, and/or otherwise increase the communicative bandwidth required by the controller of the present invention. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces may be used to engage with various communications network types. For example, multiple network interfaces may be employed to allow for the communication over broadcast, multicast, and/or unicast networks.

Input Output interfaces (I/O) may accept, communicate, and/or connect to user input devices, peripheral devices, cryptographic processor devices, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices often are a type of peripheral device (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices may be external, internal and/or part of the controller of the present invention. Peripheral devices may also include, for example, an antenna, audio devices (e.g., line-in, line-out, microphone input, speakers, etc.), cameras (e.g., still, video, webcam, etc.), drive motors, lighting, video monitors and/or the like.

Cryptographic units such as, but not limited to, microcontrollers, processors, interfaces, and/or devices may be attached, and/or communicate with the controller of the present invention. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

Memory

Generally, any mechanization and/or embodiment allowing a processor to affect the storage and/or retrieval of information is regarded as memory. However, memory is a fungible technology and resource, thus, any number of memory embodiments may be employed in lieu of or in concert with one another. It is to be understood that the controller of the present invention and/or a computer systemization may employ various forms of memory. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory will include ROM, RAM, and a storage device. A storage device may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

Component Collection

The memory may contain a collection of program and/or database components and/or data such as, but not limited to: operating system component(s) (operating system); information server component(s) (information server); user interface component(s) (user interface); Web browser component(s) (Web browser); database(s); mail server component(s); mail client component(s); cryptographic server component(s) (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device, they may also be loaded and/or stored in memory such as: peripheral devices, RAM, remote storage facilities through a communications network, ROM, various forms of memory, and/or the like.

Operating System

The operating system component is an executable program component facilitating the operation of the controller of the present invention. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millennium/NT/Vista/XP (Server), Palm OS, and/or the like. The operating system may be one specifically optimized to be run on a mobile computing device, such as iOS, Android, Windows Phone, Tizen, Symbian, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the controller of the present invention to communicate with other entities through a communications network. Various communication protocols may be used by the controller of the present invention as a subcarrier transport mechanism for interaction, such as, but not limited to: multicast, TCP/IP, UDP, unicast, and/or the like.

Information Server

An information server component is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C # and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the controller of the present invention based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port, and/or the like. An information server may communicate to and/or with other components in a component collection, including itself, and/or facilities of the like. Most frequently, the information server communicates with the database of the present invention, operating systems, other program components, user interfaces, Web browsers, and/or the like.

Access to the database of the present invention may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the present invention. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the present invention as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millennium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

Web Browser

A Web browser component is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the enabled nodes of the present invention. The combined application may be nugatory on systems employing standard Web browsers.

Mail Server

A mail server component is a stored program component that is executed by a CPU. The mail server may be a conventional Internet mail server such as, but not limited to sendmail, Microsoft Exchange, and/or the like. The mail server may allow for the execution of program components through facilities such as ASP, ActiveX, (ANSI) (Objective-) C (++), C # and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the present invnetion.

Access to the mail of the present invention may be achieved through a number of APIs offered by the individual Web server components and/or the operating system.

Also, a mail server may contain, communicate, generate, obtain, and/or provide program component, system, user, and/or data communications, requests, information, and/or responses.

Mail Client

A mail client component is a stored program component that is executed by a CPU. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

Cryptographic Server

A cryptographic server component is a stored program component that is executed by a CPU, cryptographic processor, cryptographic processor interface, cryptographic processor device, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RC5), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the present invention may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for an digital audio file. A cryptographic component may communicate to and/or with other components in a component collection, including itself, and/or facilities of the like. The cryptographic component supports encryption schemes allowing for the secure transmission of information across a communications network to enable the component of the present invention to engage in secure transactions if so desired. The cryptographic component facilitates the secure accessing of resources on the present invention and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

The Database of the Present Invention

The database component of the present invention may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the database of the present invention may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component includes several tables. A Users (e.g., operators and physicians) table may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The Users table may support and/or track multiple entity accounts. A Clients table may include fields such as, but not limited to: user_id, client_id, client_ip, client_type, client_model, operating_system, os_version, app_installed_flag, and/or the like. An Apps table may include fields such as, but not limited to: app_ID, app_name, app_type, OS_compatibilities_list, version, timestamp, developer_ID, and/or the like. A beverages table including, for example, heat capacities and other useful parameters of different beverages, such as depending on size beverage_name, beverage_size, desired_coolingtemp, cooling_time, favorite_drinker, number_of_beverages, current_beverage_temperature, current_ambient_temperature, and/or the like. A Parameter table may include fields including the foregoing fields, or additional ones such as cool_start_time, cool_preset, cooling_rate, and/or the like. A Cool Routines table may include a plurality of cooling sequences may include fields such as, but not limited to: sequence_type, sequence_id, flow_rate, avg_water_temp, cooling_time, pump_setting, pump_speed, pump_pressure, power_level, temperature sensor id number, temperature sensor location, and/or the like.

In one embodiment, user programs may contain various user interface primitives, which may serve to update the platform of the present invention. Also, various accounts may require custom database tables depending upon the environments and the types of clients the system of the present invention may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components. The system of the present invention may be configured to keep track of various settings, inputs, and parameters via database controllers.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for facilitating food preparation, comprising:
opening, using an electronic device having a graphical user interface, a digital application;
creating, using the graphical user interface, a user profile with the digital application;
selecting, using the graphical user interface, one or more grocery stores to be designated grocery stores;
selecting, using the graphical user interface, a recipe for preparation by a user;
displaying, using the graphical user interface, a list of one or more ingredients required for preparation of the recipe;
selecting, using the graphical user interface in conjunction with the digital application, one or more items in the list of the one or more ingredients required for preparation of the recipe;
ordering, using the digital application, any items selected from the one or more items in the list of the one or more ingredients required for preparation of the recipe; and
displaying, to the user, using the graphical user interface, the recipe.

2. The method as recited in claim 1, wherein the ordering further includes ordering from one or more of the designated grocery stores.

3. The method as recited in claim 1, wherein the selecting the recipe further includes selecting the recipe from a list of available recipes.

4. The method as recited in claim 3, further comprising:
searching, using the graphical user interface, through the list of available recipes.

5. The method as recited in claim 1, wherein the electronic device is selected from the group consisting of: a smart phone; a desktop computer; a laptop computer; and a tablet computer.

6. The method as recited in claim 1, wherein the displaying the recipe further includes reading, using a speaker, each step in preparing the recipe.

7. The method as recited in claim 1, wherein the ordering further includes:
selecting which store to purchase each item in the items selected from the one or more items in the list of the one or more ingredients required for preparation of the recipe.

8. The method as recited in claim 1, further comprising:
delivering, to the user, the items selected from the one or more items in the list of the one or more ingredients required for preparation of the recipe.

9. The method as recited in claim 1, wherein the ordering further includes sending, to one or more grocery stores, a list of the items selected from the one or more items in the list of the one or more ingredients required for preparation of the recipe.

10. A system for facilitating food preparation, comprising:
an electronic device,
wherein the electronic device includes:
a memory;
a processor; and
a graphical user interface having a display, and
wherein the electronic device is configured to:
open, using an electronic device having a graphical user interface, a digital application;
create, using the processor, a user profile with the digital application using information input using the graphical user interface;
receive a selection of one or more grocery stores to be designated grocery stores;
receive a selection of a recipe for preparation by a user using the graphical user interface;
display, using the graphical user interface, a list of one or more ingredients required for preparation of the recipe;
receive a selection of one or more items in the list of the one or more ingredients required for preparation of the recipe;
order any items selected from the one or more items in the list of the one or more ingredients required for preparation of the recipe; and
display, to the user, using the graphical user interface, the recipe.

11. The system as recited in claim 10, wherein the ordering further includes ordering from one or more of the designated grocery stores.

12. The system as recited in claim 10, wherein the receiving the selection of the recipe further includes receiving the selection of the recipe from a list of available recipes.

13. The system as recited in claim 12, wherein the electronic device is further configured to enable the user to search, using the graphical user interface, through the list of available recipes.

14. The system as recited in claim 10, wherein the electronic device is selected from the group consisting of: smart phone; a desktop computer; a laptop computer; a tablet computer; a smart watch, and a smart speaker.

15. The system as recited in claim 10, wherein the electronic device further includes a speaker configured to read each step in preparing the recipe.

16. The system as recited in claim 10, wherein the electronic device is further configured to receive a selection of which store to purchase each item in the items selected from the one or more items in the list of the one or more ingredients required for preparation of the recipe.

17. The system as recited in claim 10, further comprising one or more remote servers.

18. The system as recited in claim 17, wherein the electronic device is coupled to the one or more remote servers.

19. The system as recited in claim 18, wherein one or more second electronic devices are coupled to the one or more remote servers.

20. The system as recited in claim 17, wherein the one or more servers are configured to send, to one or more grocery stores, a list of the items selected from the one or more items in the list of the one or more ingredients required for preparation of the recipe.
